(19) United States
(12) Patent Application Publication
(10) Pub. No.: US 2016/0000694 A1
(43) Pub. Date: Jan. 7, 2016

(54) COMPOSITION AND SYSTEM FOR MANUFACTURING SAME

(71) Applicant: Gr8 Organics, Inc., New York, NY (US)

(72) Inventor: Robert B. Kaimowitz, New York, NY (US)

(21) Appl. No.: 14/323,429

(22) Filed: Jul. 3, 2014

(51) Int. Cl. A61K 8/97; A61Q 19/00; C11B 9/00

(52) U.S. Cl. CPC A61K 8/97; C11B 9/00; A61Q 19/007; A61K 2800/30

(57) ABSTRACT

The disclosed invention relates to a composition that can be applied to skin or scalp or dispersed into air, and systems for manufacturing the same. In one aspect, the composition includes cedarwood oil, tea tree oil, clove oil, hempseed oil, a peppermint oil and ethanol mixture, a lavender oil and ethanol mixture, a tincture created with calendula and ethanol, and a tincture created with hops, red clover, and glycerin.

### FIELD OF THE INVENTION

The disclosed invention relates to a composition that can be applied to skin or scalp or dispersed into air, and systems for manufacturing the same. In particular, the disclosed invention relates to an organic composition.

### BACKGROUND

The progress of science has created the ability to manufacture everything in vast quantities. And where the supply of naturally occurring components are insufficient, advancements in science have allowed the creation of artificially replacements. However, such artificial replacements may not provide all of the benefits of the naturally occurring components. Additionally, potential long-term, detrimental effects of artificial components may be unknown. Accordingly, there is continuing interest in providing products that are largely based on naturally-occurring components.

### SUMMARY

The disclosed invention relates to a composition that can be applied to skin or scalp or dispersed into air, and systems for manufacturing the same.

In one aspect, the composition can include cedarwood oil, tea tree oil, clove oil, hempseed oil, a peppermint oil and ethanol mixture, a lavender oil and ethanol mixture, a tincture created with calendula and ethanol, and a tincture created with hops, red clover, and glycerin.

In one embodiment, the peppermint oil and ethanol mixture is approximately 39% of the composition by weight. In one embodiment, the peppermint oil and ethanol mixture has a proportion of 10 pounds of peppermint oil to 100 gallons of ethanol.

In one embodiment, the lavender oil and ethanol mixture is approximately 21% of the composition by weight. In one embodiment, the lavender oil and ethanol mixture has a proportion of 10 pounds of lavender oil to 100 gallons of ethanol.

In one embodiment, the hempseed oil is approximately 10% of the composition by weight. In one embodiment, the tea tree oil is approximately 0.75% of the composition by weight. In one embodiment, the clove oil is approximately 0.15% of the composition by weight. In one embodiment, the cedarwood oil is approximately 0.15% of the composition by weight. In one embodiment, the first tincture is approximately 1% of the composition by weight. In one embodiment, the second tincture is approximately 2.5% of the composition by weight.

In one aspect, the composition can include cedarwood oil, tea tree oil, clove oil, hempseed oil, peppermint oil, lavender oil, a first tincture created with calendula, and a second tincture created with hops and red clover. In one embodiment, the cedarwood oil, tea tree oil, clove oil, and hempseed oil, are essential oils. In one embodiment, the first tincture is further created with ethanol. In one embodiment, the second tincture is further created with glycerin. In one embodiment, the composition can further include shea butter.

In one aspect, the composition can include extracts from hops, red clover, calendula, cedarwood, tea tree, clove, hempseed, peppermint, and lavender. In one embodiment, the composition can further include ethanol and glycerin. In one embodiment, the composition can further include shea butter.

These aspects and embodiments are exemplary and do not limit the scope of the disclosed technology, which will be apparent from a reading of the following detailed description and the associated drawings.

### BRIEF DESCRIPTION OF THE DRAWINGS

FIG. 1 is a diagram of a composition and an exemplary system for manufacturing the composition;

FIG. 2 is a diagram of one component of the composition of FIG. 1;

FIG. 3 is a diagram of another component of the composition of FIG. 1; and

FIG. 4 is a diagram of yet another component of the composition of FIG. 1.

### DETAILED DESCRIPTION

The disclosed invention relates to a composition that can be applied to skin or scalp or dispersed into air, and systems for manufacturing the same. As described below herein, the composition can be applied to skin or scalp irritated by itchiness and/or inflammation, including but not limited to application to eczema, scalp-itch, jock itch and insect bites.

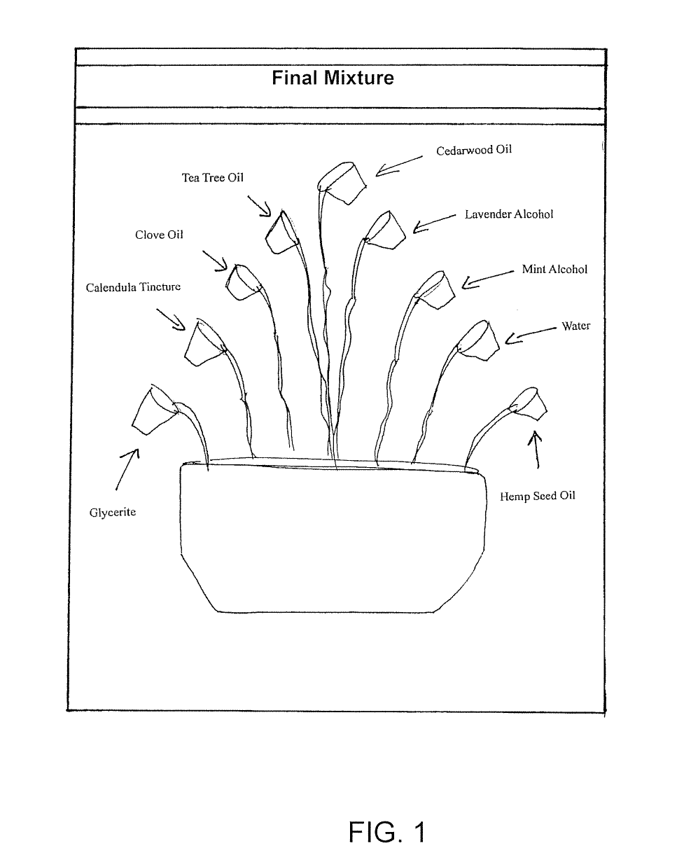

Referring now to FIG. 1, there is shown a diagram of an exemplary composition. In the illustrated embodiment, the composition includes cedarwood oil, tea tree oil, clove oil, and hempseed oil. In one embodiment, these oils can be what is known as “essential oils”, which are plant oils extracted from plants. In various embodiments, essential oil can be extracted from a plant by pressing a fresh plant, by solvents, and/or by steam distillation, which will be known to those skilled in the art.

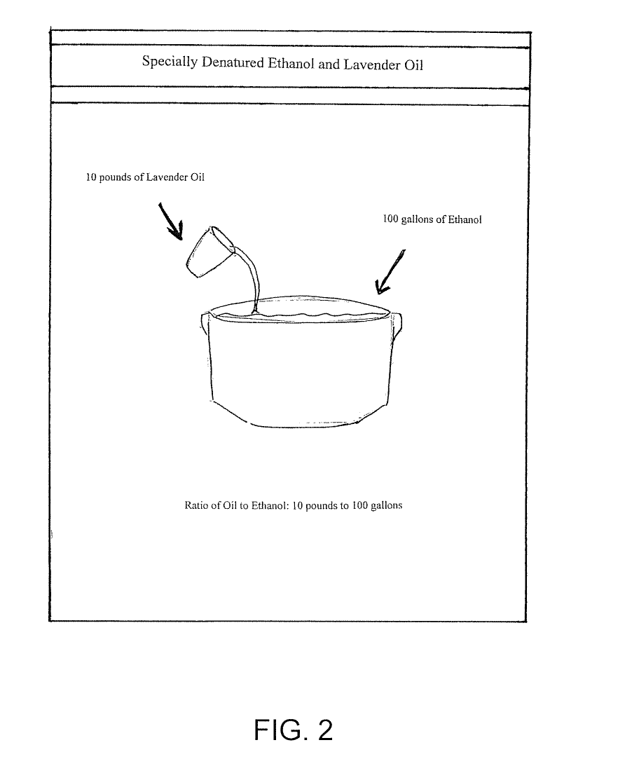

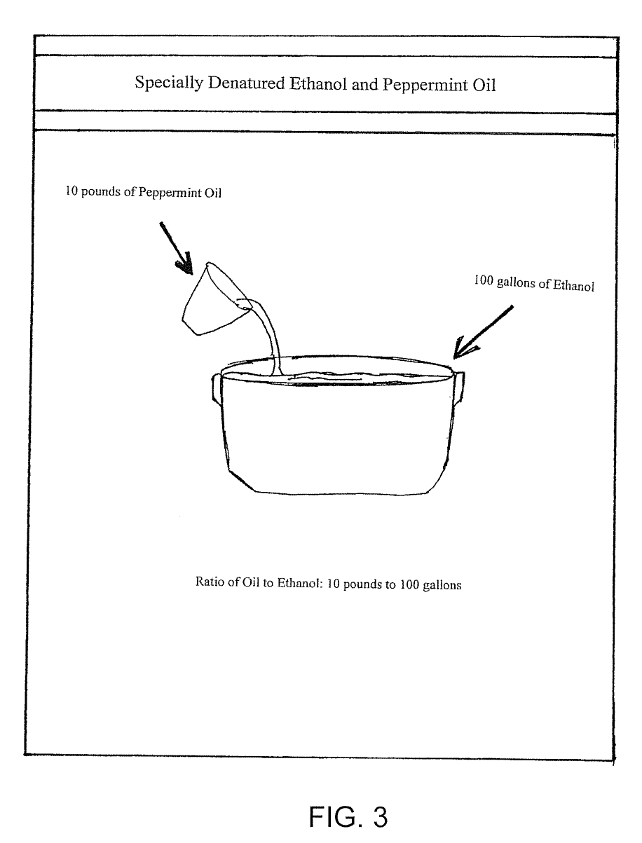

With continuing reference to FIG. 1, the composition also includes two mixtures of alcohol and essential oils: peppermint alcohol and lavender alcohol. In one embodiment, the peppermint alcohol can be a mixture of peppermint oil and ethanol, and the lavender alcohol can be a mixture of lavender oil and ethanol. In one embodiment, the peppermint oil and lavender oil can be essential oils. In various embodiments, the alcohol can be an alcohol other than ethanol. The peppermint alcohol and the lavender alcohol are described in more detail in connection with FIG. 2 and FIG. 3.

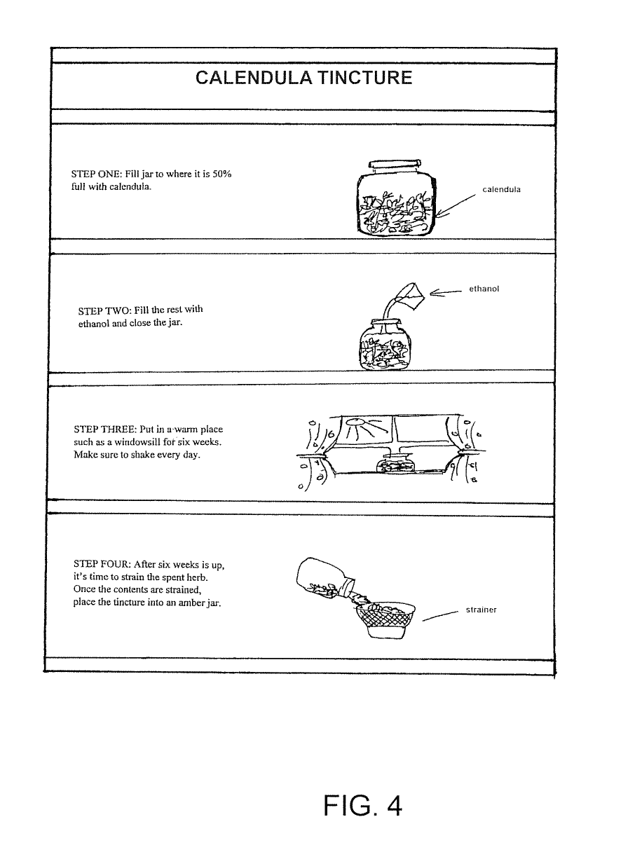

With continuing reference to FIG. 1, the composition also includes two tinctures: a hops and red clover tincture (shown as “glycerite”) and a calendula tincture. As used herein, a tincture is a solution created by soaking a plant in a fluid for a period of time and then removing the plant after the period of time. In one embodiment, the fluid can be ethanol. In one embodiment, the fluid can be glycerin. The tinctures are described in more detail in connection with FIG. 4

With continuing reference to FIG. 1, the composition also includes water, and the components are mixed together to form the disclosed composition. Accordingly, in view of the description above, the composition can include cedarwood oil, tea tree oil, clove oil, hempseed oil, peppermint oil, lavender oil, and tea tree oil, which can be essential oils. In one aspect, the composition can include extracts from or concentrations of hops, red clover, and calendula.

In one aspect, the composition can include extracts from or concentrations of hops, red clover, calendula, cedarwood, tea tree, clove, hempseed, peppermint, and lavender. Each of these plants has beneficial qualities and characteristics, such as:
Hemp seed—anti-inflammatory, healing, conditions, moisturizes, strengthens skin
Calendula—anti-inflammatory, moisturizing
Tea tree—antiseptic, antibacterial, pain/itch relief, antifungal, dandruff
Lavender—soothing, calming, antiseptic, antifungal, dandruff
Clove—antioxidant, anti-inflammatory, aides concentration, cooling
Cedarwood—anxiety, astringent, dandruff, aids circulation
Red clover—anti-inflammatory
Hops—healing, aids circulation
Peppermint—soothing, anti-inflammatory

Because of the illustrative benefits above, the disclosed composition has a variety of applications for skin and scalp and for dispersion into air.

In one embodiment, the disclosed composition can be applied to eczema skin and scalp conditions. In one embodiment, the composition can be applied as a spray onto the skin or scalp. The benefits of the components and the spray delivery can noticeably and rapidly soothe the itch and inflammation of eczema, usually within hours of application.

In one embodiment, the disclosed composition can be applied to the scalp. In one embodiment, the composition can be applied with an applicator cap. The benefits of the components and the applicator cap delivery can sooth scalp itch and dermatitis of the scalp, and can heal the cause of dandruff.

In one embodiment, the disclosed composition can be applied to insect bites or small cuts and scrapes of the skin. In one embodiment, the composition can be applied as a spray onto the skin. The essential oils aid in healing and soothing of scrapes and small cuts. Because the composition includes alcohol from ethanol and/or glycerin, the disclosed composition can also provide a disinfecting benefit.

In one embodiment, the disclosed composition can be dispersed into the air as a spray, and can freshen the air and/or remove smoke odors.

Accordingly, what have been described above are components for the disclosed composition, and their benefits and applications. The illustrated uses and applications are merely exemplary and do not limit the scope of the disclosed composition and systems and their uses. In certain embodiments, the peppermint can be replaced with another mint. Additional aspects of the composition are described below.

Referring now to FIG. 2, there is shown one embodiment of the lavender alcohol in FIG. 1. In the illustrated embodiment, the alcohol can be ethanol, and the lavender oil can be an essential oil. The proportion of lavender oil to ethanol is about 10 pounds of lavender oil to 100 gallons of ethanol. The amount of these components can be another number, while maintaining these proportions. The lavender oil and the ethanol can be mixed together to form the lavender alcohol component. The illustrated embodiment is merely exemplary. In other embodiments, the alcohol can be different from ethanol, and the lavender oil need not be an essential oil. In other embodiments, the proportion of lavender oil to alcohol can be another proportion.

Referring now to FIG. 3, there is shown one embodiment of the peppermint alcohol in FIG. 1. In the illustrated embodiment, the alcohol can be ethanol, and the peppermint oil can be an essential oil. The proportion of peppermint oil to ethanol is about 10 pounds of lavender oil to 100 gallons of ethanol. The amount of these components can be another number, while maintaining these proportions. The peppermint oil and the ethanol can be mixed together to form the peppermint alcohol component. The illustrated embodiment is merely exemplary. In other embodiments, the alcohol can be different from ethanol, and the peppermint oil need not be an essential oil. In other embodiments, the proportion of peppermint oil to alcohol can be another proportion. In certain embodiments, the peppermint can be replaced with another mint.

Referring now to FIG. 4, there is shown a system of creating the calendula tincture component of FIG. 1. Although the illustrated embodiment refers to calendula, the disclosed system is applicable to other tinctures, such as a hops and red clover tincture. Referring to FIG. 4, the tincture can be created using any container, such as a jar. The container can be filled approximately half-way by volume with the calendula or another plant. Then, the container can be filled with a fluid such as ethanol, glycerin, or another alcohol. The container can be closed and placed in a warm environment for a period of time, such as six weeks. The solution can be stimulated on a regular basis, such as by shaking the container once a day. Upon reaching the period of time, the container can be opened and the plant can be removed, such as by using a strainer. The remaining liquid is the tincture.

The hoops and red clover tincture (shown as “glycerite”) of FIG. 1, can be created using the illustrative system in FIG. 4. For this tincture, the fluid can be glycerin.

The disclosed system is merely exemplary and does not limit the scope of the disclosed composition and system. For example, the container can be any container that is properly sized for the desired manufacturing capacity. The container need not be filled half-way for every application and can be filled to another volume depending on the application. Additionally, the warm environment can be created by directly heating the container or by heating the container's surroundings, using any heat source appropriate for the manufacturing. Stimulation of the container can be performed by machines moving the entire container and/or by implements moving inside the container, such as a stirrer. Lastly, the duration for preparing the tincture can vary depending on the application and the manufacturing technology that is applied. For example, certain manufacturing technologies may require much less time than six weeks to produce the tincture.

The following will describe exemplary proportions for the disclosed composition. In one embodiment, the proportions by weight can be approximately the following:
Peppermint alcohol: 39.00% by weight
Lavender alcohol: 21.00% by weight
Hempseed oil: 10.00% by weight
Hops and red clover tincture (“glycerite”): 2.5% by weight
Tea tree oil: 0.75% by weight
Clove oil: 0.15% by weight
Calendula tincture: 1.00% by weight
Cedarwood oil: 0.15% by weight
Water: 25.45% by weight

These proportions are exemplary and do not limit scope of the disclosed composition and systems. Other proportions are contemplated to be within the scope of the disclosed technology. For example, another proportion of components can be:
Peppermint alcohol: 39.00% by weight
Lavender alcohol: 21.00% by weight
Hempseed oil: 10.00% by weight
Hops and red clover tincture (“glycerite”): 2.5% by weight
Tea tree oil: 1.5% by weight
Clove oil: 0.15% by weight
Calendula tincture: 1.00% by weight
Cedarwood oil: 0.15% by weight
Water: 24.70% by weight

In one aspect of the disclosed invention, the alcohols used in the composition can be replaced with a thicker-consistency alternative, such as a cream or butter. In one embodiment, the cream or butter can be shea butter. Accordingly, the shea butter can be mixed with the peppermint oil or mixed with the lavender oil. The shea butter is exemplary, and other butters or thicker-consistency carriers can be used. The thicker consistency carrier can allow the composition to be applied to skin or scalp as a lotion or cream.

In one aspect of the disclosed invention, the composition can include five minimum components: mint, lavender, hempseed, clove, and a tincture created with hops and red clover. In one embodiment, the composition can include extracts of the plants of these components.

In one aspect of the disclosed invention, the composition can include seven minimum components: mint, lavender, hempseed, clove, tea tree, a tincture created with hops and red clover, and a tincture created with calendula. In one embodiment, the composition can include extracts of the plants of these components.

The illustrations and embodiments disclosed herein are exemplary and do not limit the spirit and scope of the disclosed system and composition. Combinations of one or more disclosed embodiments or portions of one or more embodiments are contemplated as being within the scope of the disclosed system and composition.

What is claimed is:

1. A composition comprising:
cedarwood oil;
tea tree oil;
clove oil;
hempseed oil;
a peppermint oil and ethanol mixture;
a lavender oil and ethanol mixture;
a first tincture created with calendula and ethanol; and
a second tincture created with hops, red clover, and glycerin.

2. The composition of claim 1, wherein the peppermint oil and ethanol mixture is approximately 39% of the composition by weight.

3. The composition of claim 2, wherein the peppermint oil and ethanol mixture has a proportion of 10 pounds of peppermint oil to 100 gallons of ethanol.

4. The composition of claim 1, wherein the lavender oil and ethanol mixture is approximately 21% of the composition by weight.

5. The composition of claim 4, wherein the lavender oil and ethanol mixture has a proportion of 10 pounds of lavender oil to 100 gallons of ethanol.

6. The composition of claim 1 wherein the hempseed oil is approximately 10% of the composition by weight.

7. The composition of claim 1 wherein the second tincture is approximately 2.5% of the composition by weight.

8. The composition of claim 1 wherein the tea tree oil is approximately 0.75% of the composition by weight.

9. The composition of claim 1 wherein the clove oil is approximately 0.15% of the composition by weight.

10. The composition of claim 1 wherein the first tincture is approximately 1% of the composition by weight.

11. The composition of claim 1 wherein the cedarwood oil is approximately 0.15% of the composition by weight.

12. A composition comprising:
cedarwood oil;
tea tree oil;
clove oil;
hempseed oil;
peppermint oil;
lavender oil;
a first tincture created with calendula; and
a second tincture created with hops and red clover.

13. The composition of claim 12, wherein the cedarwood oil, tea tree oil, clove oil, and hempseed oil, are essential oils.

14. The composition of claim 12, wherein the first tincture is further created with ethanol.

15. The composition of claim 12, wherein the second tincture is further created with glycerin.

16. The composition of claim 12, further comprising shea butter.

17. A composition comprising extracts from hops, red clover, calendula, cedarwood, tea tree, clove, hempseed, peppermint, and lavender.

18. The composition of claim 17, further comprising ethanol and glycerin.

19. The composition of claim 17, further comprising shea butter.
